(19) United States
(12) Patent Application Publication
(10) Pub. No.: US 2025/0261844 A1
(43) Pub. Date: Aug. 21, 2025

(54) APPARATUS FOR PROVIDING TRAINING DURING LAPAROSCOPIC SURGERY

(71) Applicants: HENRY FORD HEALTH SYSTEM, Detroit, MI (US); LAPARASSIST, INC., Lansing, MI (US)

(72) Inventors: Kay Elaine HADRICK, East Lansing, MI (US); Maxwell Claef HAKUN, East Lansing, MI (US); Colin Thomas O'HERN, Lansing, MI (US); Simon Wolfgang SANCHEZ, East Lansing, MI (US); Annmarie Lyn VILKINS, Detroit, MI (US)

(73) Assignees: HENRY FORD HEALTH SYSTEM, Detroit, MI (US); LAPARASSIST, INC., Lansing, MI (US)

(21) Appl. No.: 18/856,962

(22) PCT Filed: Apr. 24, 2023
(86) PCT No.: PCT/US2023/019556

§ 371 Date: Oct. 15, 2024

Related U.S. Application Data
(60) Provisional application No. 63/333,645, filed on Apr. 22, 2022.

(51) Int. Cl. A61B 1/313; G06F 3/033; G09B 23/28

(52) U.S. Cl. CPC A61B 1/3132; G06F 3/033; G09B 23/285

(57) ABSTRACT

An apparatus for providing training for a learner by a teacher during laparoscopic surgery includes a housing including a first glasses arm, the housing configured to be mounted to a pair of glasses by the first glasses arm. A light source, such as a laser pointer, is mounted on the housing and configured to generate a light beam. A control module is disposed within the housing in electrical communication with the light source, the control module including a power source for providing power to the light source. When the pair of glasses is worn by the teacher, a direction of the light beam generated by the light source corresponds to a head position of the teacher to indicate a location for the learner. The apparatus may further include a foot pedal in wireless communication with the control module for hands-free control of the light source.

### CROSS-REFERENCE TO RELATED APPLICATIONS

This application claims the benefit of U.S. provisional application Ser. No. 63/333,645 filed Apr. 22, 2022, the disclosure of which is hereby incorporated in its entirety by reference herein.

### TECHNICAL FIELD

Embodiments relate to an apparatus for providing training for a learner by a teacher during surgery, such as laparoscopy surgery.

### BACKGROUND

Laparoscopic surgery is a minimally invasive surgical technique in which narrow tubes, known as trocars, are inserted into the patient through small incisions (ports). Surgeons achieve an intra-abdominal view by insufflating the abdomen with CO2 gas and inserting a laparoscope (camera) through a trocar. The intra-abdominal view is projected on a video monitor on top of a laparoscopic tower where surgeons visualize anatomical features and positioning of operating instruments.

The laparoscope and operating instruments are controlled by the hands of two surgeons: the teacher and the learner. Traditionally, laparoscopic surgeries utilize three to four ports that provide intra-abdominal access to the laparoscope and operating instruments. During laparoscopic operations, there are occurrences when the teacher would like to communicate exactly where the learner should intervene (e.g., grasp, suture, cauterize, etc.) but verbal communication does not sufficiently describe the desired location. Since the hands of the teacher and learner are occupied with the laparoscope and operating instruments, pointing cannot be used as an alternative method. This communication issue at minimum slows the progression of the surgery and at maximum, leads to more complications, such as inadvertent injury to surrounding anatomical structures or hemorrhage in instances in which the learner is unclear of the intended next step.

### SUMMARY

In one or more embodiments, an apparatus for providing training for a learner by a teacher during laparoscopic surgery includes a housing including a first glasses arm, the housing configured to be mounted to a pair of glasses by the first glasses arm. A light source is mounted on the housing and configured to generate a light beam, and a control module is disposed within the housing in electrical communication with the light source, the control module including a power source for providing power to the light source. When the pair of glasses is worn by the teacher, a direction of the light beam generated by the light source corresponds to a head position of the teacher to indicate a location for the learner.

In one or more embodiments, an apparatus for providing training for a learner by a teacher during laparoscopy surgery includes a pair of glasses including a lens portion and a frame portion surrounding the lens portion. The apparatus further includes a housing including a first glasses arm, the housing configured to be mounted to the frame portion by the first glasses arm. A laser pointer is mounted on the housing and configured to generate a light beam, and a control module is disposed in a first cavity within the housing in electrical communication with the laser pointer, the control module including a power source for providing power to the laser pointer. The pair of glasses is arranged to be worn by the teacher such that a direction of the light beam generated by the laser pointer corresponds to a head position of the teacher to indicate a location for the learner.

In one or more embodiments, an apparatus for providing training for a learner by a teacher during laparoscopic surgery includes a pair of glasses including a lens portion and a frame portion surrounding the lens portion. The apparatus further includes a housing including a first glasses arm having a front mounting portion with mounting pins extending therefrom for removably and pivotally mounting the housing to the frame portion. A laser pointer is mounted on the first glasses arm and configured to generate a light beam, and a control module is disposed within a first cavity of the housing in electrical communication with the laser pointer, the control module including a power source for providing power to the laser pointer. The apparatus further includes a foot pedal in wireless communication with the control module for hands-free control of the laser pointer. The pair of glasses is arranged to be worn by the teacher such that a direction of the light beam generated by the laser pointer corresponds to a head position of the teacher to indicate a location for the learner.

In one or more embodiments, a switch is disposed within the housing in electrical communication with the light source. In one or more embodiments, the first glasses arm includes an upper mounting portion including at least one tab having a channel configured to at least partially receive the light source. In one or more embodiments, the housing includes a hollow projection member adjacent the upper mounting portion and including a conduit connected to the first cavity for routing wiring from the laser pointer to the first cavity. In one or more embodiments, the apparatus includes a cover removably connectable to the housing for enclosing the first cavity. In one or more embodiments, the apparatus includes a head strap arranged to be connected to the pair of glasses.

### BRIEF DESCRIPTION OF THE DRAWINGS

FIG. 1 is a photograph illustrating a teacher instructing a learner while their hands are occupied with instrumentation during laparoscopic surgery;

FIG. 2 is a photograph of a video monitor showing instrumentation held by the teacher's hands and the learner's hands during laparoscopic surgery;

FIG. 3 is a photograph illustrating how an apparatus according to one or more embodiments functions during laparoscopic surgery;

FIG. 4 is a photograph of a video monitor showing instrumentation held by the teacher's hands and the learner's hands with the apparatus disclosed herein in use during laparoscopic surgery;

FIG. 5 is a photograph of the apparatus according to one or more embodiments, including a housing with a laser pointer and a control module mounted therein, the housing mounted to a pair of glasses;

FIG. 6 is a photograph of the apparatus according to one or more embodiments;

FIG. 7 is a perspective view of a housing according to one or more embodiments;

FIG. 8 is a perspective view of a housing with a cover thereon according to one or more embodiments;

FIG. 9 is a top view of the housing showing exemplary dimensions according to one or more embodiments;

FIG. 10 is a rear view of the housing showing exemplary dimensions according to one or more embodiments;

FIG. 11 is a side view of the housing with a first glasses arm showing exemplary dimensions according to one or more embodiments;

FIG. 12 is a photograph showing an inverted view of the housing with the cover separated from the housing according to one or more embodiments;

FIG. 13 is a photograph showing an inverted view of the housing with the cover assembled according to one or more embodiments;

FIG. 14 is a photograph of the apparatus and a foot pedal for wireless communication with the control module according to one or more embodiments;

FIG. 15 is an exemplary circuit diagram for the foot pedal according to one or more embodiments;

FIG. 16 is an exemplary circuit diagram for the control module, including the microcontroller, switch, and laser pointer according to one or more embodiments;

FIG. 17 are graphs illustrating the results of a maze task using the disclosed apparatus, a conventional handheld laser pointer, and a computer mouse for academic personnel;

FIG. 18 is a graph illustrating the results of target practice using the disclosed apparatus, a conventional handheld laser pointer, and a computer mouse for academic personnel;

FIG. 19 are graphs illustrating the results of a maze task using the disclosed apparatus, a conventional handheld laser pointer, and a computer mouse for a surgical resident; and

FIG. 20 is a graph illustrating the results of target practice using the disclosed apparatus, a conventional handheld laser pointer, and a computer mouse for a surgical resident.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

According to one or more embodiments, an apparatus 10 is provided which allows surgeons (“teachers”) to accurately guide surgical trainees (“learners”) when their hands are occupied with instrumentation during surgery to expedite communication, increase surgical precision, and reduce operating room time. In one example, the apparatus 10 may be used during laparoscopic procedures in which the site of surgery is displayed on a video monitor 12.

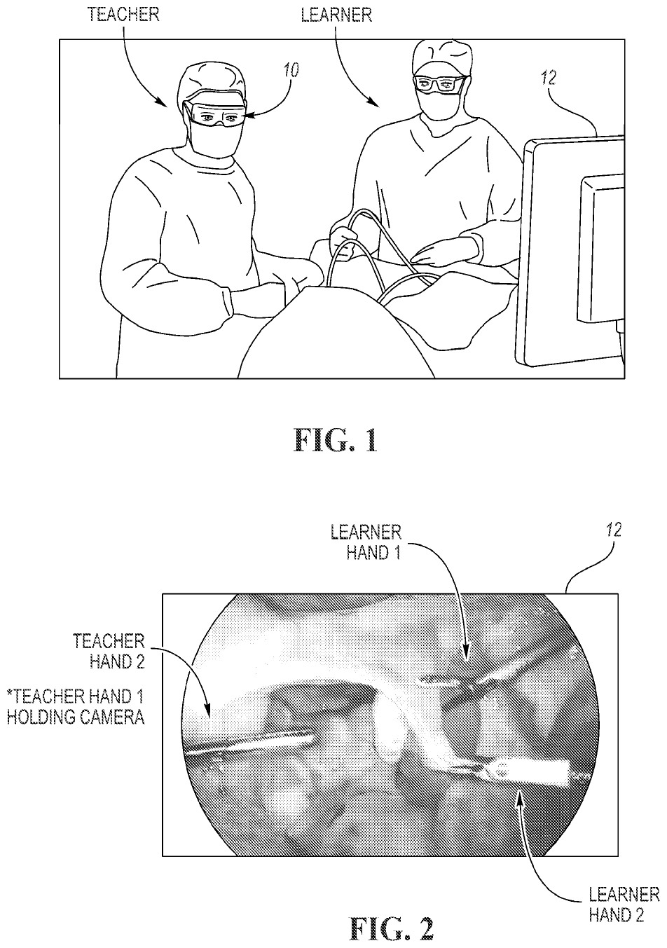

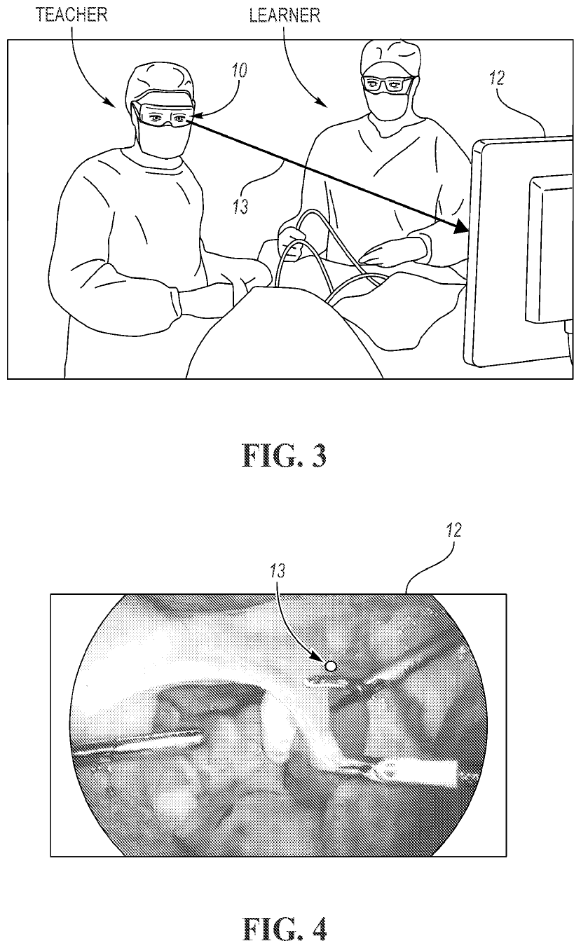

FIG. 1 is a photograph illustrating a teacher instructing a learner while their hands are occupied with instrumentation during laparoscopic surgery, and FIG. 2 is a photograph of a video monitor 12 showing instrumentation held by the teacher's hands and the learner's hands. Using the apparatus 10 disclosed herein, FIG. 3 is a photograph schematically illustrating how the apparatus 10 functions during laparoscopic surgery to direct a light beam 13 toward the video monitor 12 based on the head position of the teacher, and FIG. 4 is a photograph of a video monitor 12 showing instrumentation held by the teacher's hands and the learner's hands during laparoscopic surgery, wherein the apparatus 10 is in use to generate a light beam 13 to indicate a desired position on the video monitor 12.

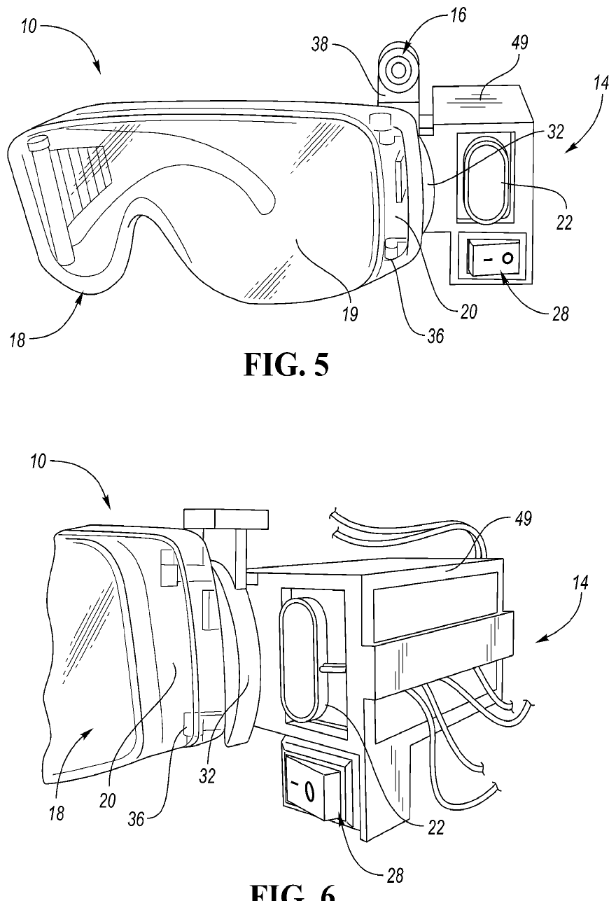

Turning now to FIGS. 5-6, the apparatus 10 includes a housing 14 for receiving and mounting a light source, such as a laser light source or laser pointer 16 (e.g., Quarton Laser Module VLM-520-73-LPT, Direct Green DOT Laser Module 3˜6V). In one or more embodiments, the apparatus 10 may include a pair of safety goggles or a pair of glasses 18 (e.g. Maxjuli Goggles Labe Safety Glasses, Over the Glasses Design and Anti-Fog UV Protection Work Goggles ANSI Z87, clear lens) onto which the housing 14 is mounted, wherein the pair of glasses 18 may include a lens portion 19 and a frame portion 20 surrounding the lens portion 19. As shown, in one embodiment the housing 14 is arranged to be mounted on the frame portion 20 of the pair of glasses 18 such that the laser pointer 16 may be located near the temple region of the wearer when in use. In this position, the laser pointer 16 may be generally aligned with the wearer's (teacher's) line of sight, such that the position of the resultant light beam is controlled by movement of the teacher's head. The power of the laser pointer 16 may be selected for the best balance of brightness of the light beam and safety during the surgical procedure.

Operation of the laser pointer 16 is controlled by a control module 22 arranged to be mounted within the housing 14. An exemplary circuit design of the control module 22 is illustrated in FIG. 16. In one or more embodiments, the control module 22 includes a microcontroller 24 in electrical communication with the laser pointer 16, the microcontroller 24 including a wireless receiver, and a power source such as a battery 26 (e.g., 9V). The control module 22, and thus the laser pointer 16, may be controlled by a switch 28, such as a toggle on/off type switch. The wearer (teacher) can initially toggle the switch 28 to the ON position, and then subsequent on/off control of the laser pointer 16 may be controlled via a remote switch, such as a foot pedal 30, for hands-free control as described further below.

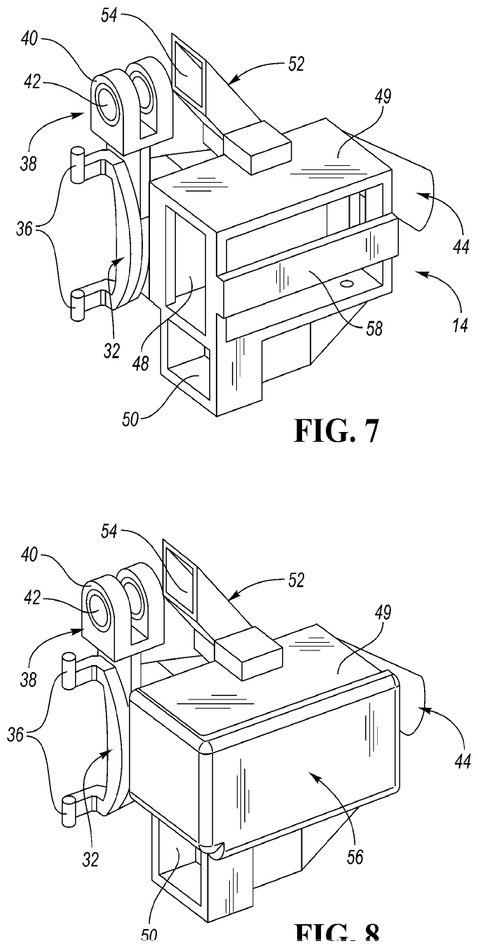

With reference now to FIGS. 7-8, perspective views of the housing 14 are illustrated. The housing 14 includes a front mounting portion 32 including upper and lower mounting pins 36 extending therefrom for removably and pivotally mounting the housing 14 to the pair of glasses 18, wherein the mounting pins 36 are configured to fit into the frame portion 20 to function as a hinge point between the housing 14 and the pair of glasses 18 (see FIG. 5).

In one or more embodiments, an upper mounting portion 38 extends upwardly from the front mounting portion 32 and includes at least one tab 40 with a channel 42 therethrough. The channel 42 is configured to at least partially receive the laser pointer 16 for mounting the laser pointer 16 on the housing 14 at approximately eye level of the wearer (teacher), such that the generated light beam may be directed according to the wearer's head position and/or line of sight. In the embodiment shown in FIGS. 7-8, two spaced tabs 40 are provided which may receive the laser pointer 16 through their respective channels 42 to securely mount the laser pointer 16 via an interference fit. Of course, alternative structures for mounting the laser pointer 16 to the housing 14 and/or to the pair of glasses 18 are also contemplated.

In one or more embodiments, the housing 14 may include (e.g., connected to or integrally formed with) a first glasses arm 44 for the pair of glasses 18, with the front mounting portion 32 provided at a proximal end of the first glasses arm 44 and the upper mounting portion 38 extending upwardly from the first glasses arm 44. With this configuration, the housing 14 is in a form that is ready to mount to the frame portion 20 of any suitable pair of glasses 18. As shown in FIG. 14, a second glasses arm 46 for the opposite side of the frame portion 20 can be provided to generally match the first glasses arm 44. Alternatively, the pair of glasses 18 may include existing arms onto which the housing 14 could be configured to be mounted.

The control module 22, including the microcontroller 24 and battery 26, may be mounted in a first cavity 48 of an outboard portion 49 the housing 14 disposed outward from the front mounting portion 32 and the first glasses arm 44. A second cavity 50 within the outboard portion 49 of the housing 14, which may be located beneath the first cavity 48, is configured to receive and mount the switch 28 so that it is accessible to the wearer for initially powering on the laser pointer 16, as explained below. All wiring associated with the control module 22 and the switch 28 can conveniently be stored within the first cavity 48 and the second cavity 50 of the housing 14, wherein the first cavity 48 may accessible via the second cavity 50. The housing 14 may further include a hollow projection member 52 positioned adjacent to the upper mounting portion 38, the projection member 52 including a conduit 54 connected to the first cavity 48 for routing wiring from the laser pointer 16 to the first cavity 48 so that the wiring is neatly contained within the housing 14.

With reference to FIGS. 8 and 12, a cover 56 may be provided to enclose the housing 14, such as the first cavity 48 once the control module 22 is mounted therein. In one or more embodiments, the cover 56 may have a generally L-shaped configuration as shown. The cover 56 may be configured to be removably connected to the housing 14 in any manner, such as via a track 58 protruding from the housing 14 along the first cavity 48 which is received by a corresponding groove 60 provided on an inner side 62 of the cover 56. Such a configuration allows for translation of the cover 56 along the housing 14 to at least partially enclose the first cavity 48 and protect the components and internal circuitry disposed therein.

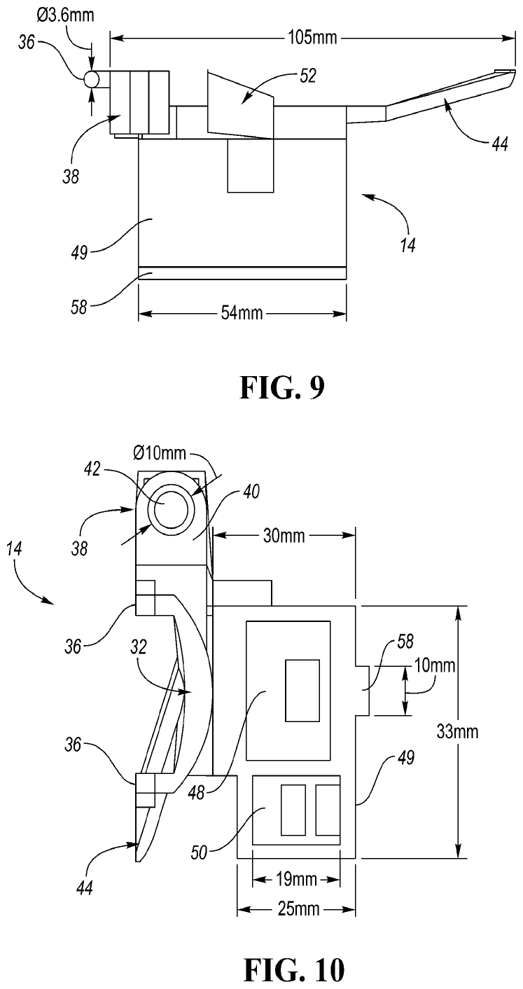

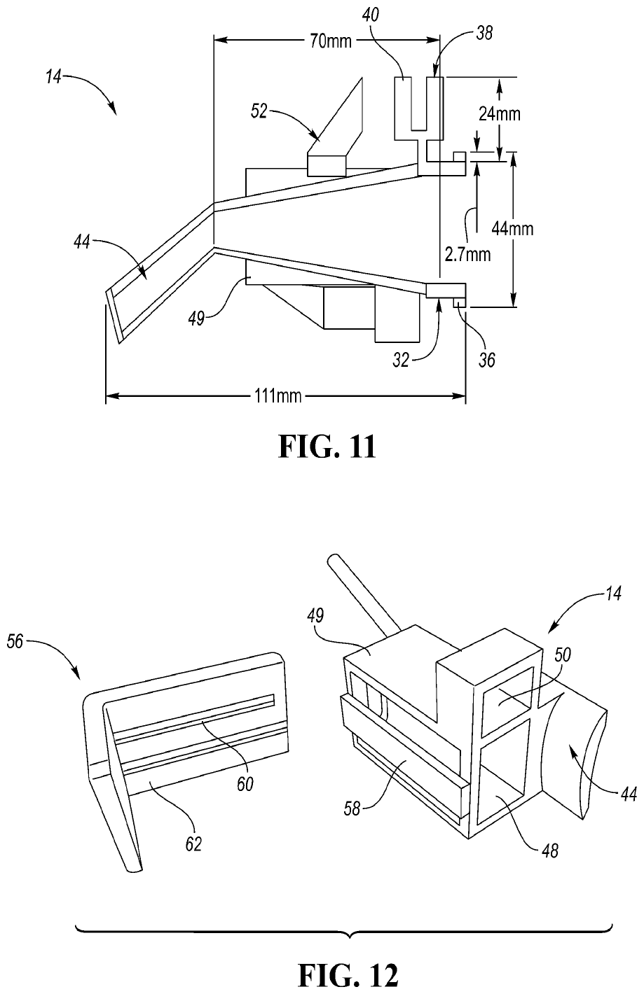

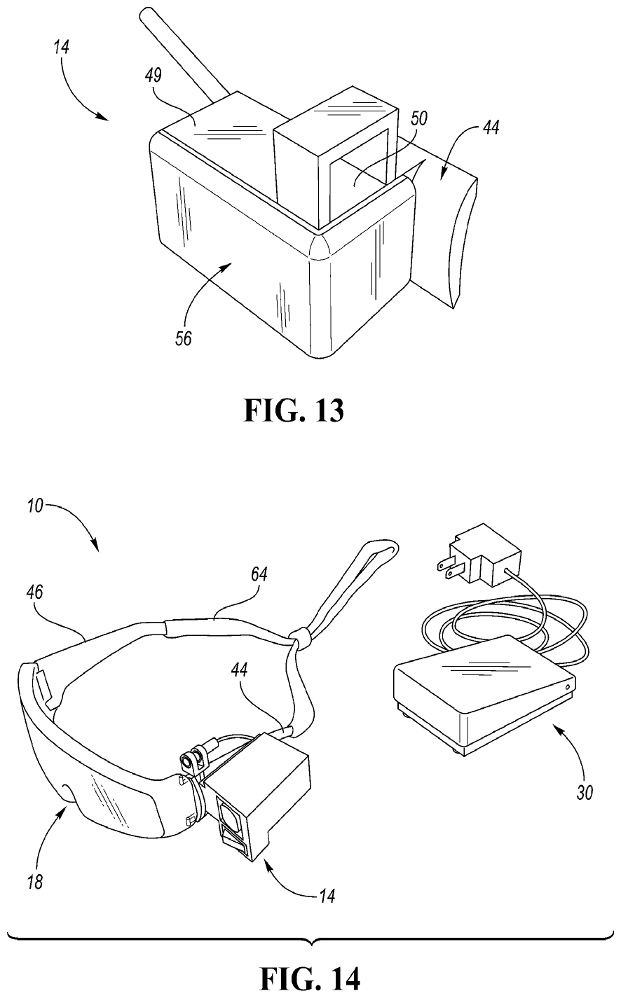

FIGS. 9-11 further illustrate the housing 14 and provide exemplary, non-limiting dimensions (e.g., in mm). FIGS. 12-13 are photographs of the housing 14 and the cover 56 in an inverted position.

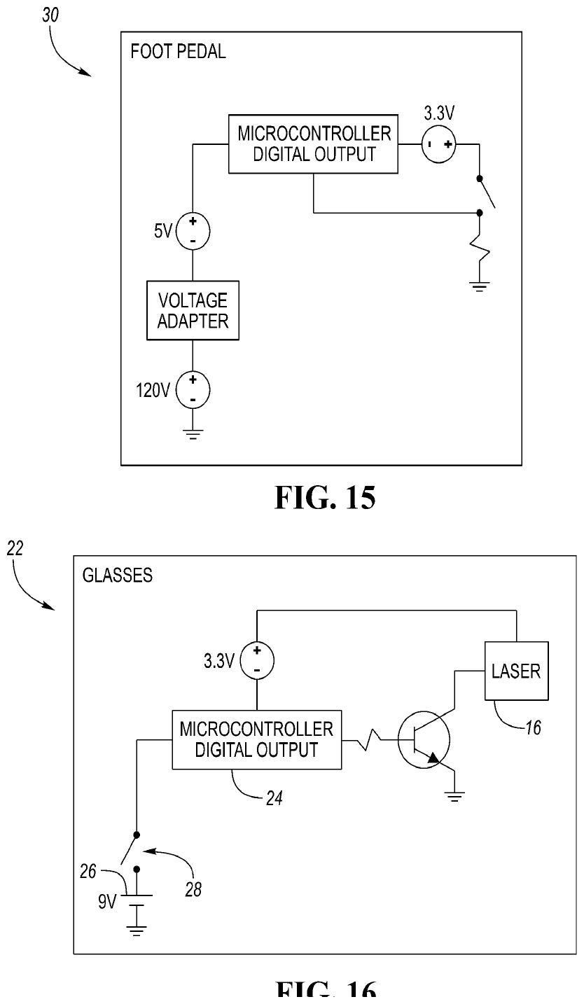

As mentioned above, a remote switch such as a foot pedal 30 (e.g., XURUI Momentary Foot Switch XF-1, Single Pedal, 10A 250V; FIG. 14) may be provided in wireless (e.g. Bluetooth) communication with the control module 22 for controlling the on/off state of the laser pointer 16. An exemplary circuit design of the foot pedal 30 is illustrated in FIG. 15, where the foot pedal 30 includes a microcontroller with a wireless transmitter.

In operation, the “teacher” may wear the pair of glasses 18 and operate the foot pedal 30 during laparoscopic surgery to activate the laser pointer 16 toward the video monitor 12, allowing for effective communication with the “learner”. In one non-limiting embodiment, a brief time delay may be provided in the control scheme of turning on the laser pointer 16 after the foot pedal 30 is pressed, such as to diminish the possibility of any laser-related injuries.

In one or more embodiments, the apparatus 10 may operate as follows during activation of the laser pointer 16: 1) the teacher turns the laser pointer 16 on via the switch 28; 2) the teacher engages the foot pedal 30 and applies force with his/her foot; 3) the foot pedal 30 powers on the wireless transmitter (e.g. Bluetooth) associated with the foot pedal 30; 4) the wireless transmitter pairs with the wireless receiver associated with the control module 22; 5) pairing of the wireless transmitter and the wireless receiver triggers an ON state for the laser pointer 16 (i.e., by outputting voltage to a pin); and 6) the teacher's head positioning directs the laser pointer 16 to generate a light beam which designates a point of interest on the video monitor 12.

In one or more embodiments, the apparatus 10 may operate as follows during deactivation of the laser pointer 16: 1) the teacher disengages the foot pedal 30 by lifting his/her foot to remove force; 2) power to the wireless transmitter is ceased; 3) unpairing between the wireless transmitter and the wireless receiver occurs; and 4) unpairing of the wireless transmitter and the wireless receiver triggers a swap to an OFF state of the laser pointer 16 (i.e., removing voltage to the laser pointer 16).

As shown in FIG. 14, a head strap 64 may be provided that is connected to the pair of glasses 18, such as to the first glasses arm 44 and the second glasses arm 46, and is configured to be tightened around the wearer's head when worn. The head strap 64 may help ensure that the pair of glasses 18 does not become unbalanced or fall during head movement by distributing the weight of the apparatus 10 across the wearer's head. In one or more embodiments, a surgical headlamp (not shown) could also be incorporated into the apparatus 10.

As described above, using the apparatus 10, the light beam generated by the laser pointer 16 may be directed to a desired area of a video monitor 12 without requiring the use of the teacher's hands. In addition, the teacher can direct the laser pointer 16 towards the video monitor 12 to non-verbally identify an anatomical structure or location for an assistant or student (learner). Information may thus be conveyed between the teacher and the learner by directing the light beam from the laser pointer 16 onto the video monitor 12 without the need for the surgeon to give a verbal description of the surgical site.

Advantageously, the apparatus 10 disclosed herein does not require use of the teacher's hands to operate the switch 28 on the control module 22, instead providing the foot pedal 30 or other remote switch in wireless communication with the control module 22 for hands-free activation and deactivation of the laser pointer 16. With this configuration, the apparatus 10 also avoids having a wired remote switch which would need to be plugged into the control module 22, as this would interfere with the sterile field in an operating room. In addition, the control module 22 disclosed herein may be entirely contained in the housing 14 mounted to the pair of glasses 18, rather than being separate from the pair of glasses 18 and needing to be stored somewhere secure during surgery, The wireless communication between the foot pedal 30 and the control module 22 also avoids the necessity of any wires which could restrict the surgeon's head/neck movement while operating. The apparatus 10 disclosed herein requires the use of only a single, existing video monitor 12 for a surgical teacher to communicate with the learner, thereby controlling costs and simplifying setup and operation of the apparatus 10. The apparatus 10 disclosed herein also does not rely on calibration to function properly, thus enhancing the reliability of the apparatus 10 in an operating room.

A study was performed to evaluate the efficacy of the apparatus 10 disclosed herein, including whether the apparatus 10 is beneficial to medical trainees and performs non-inferiorly to standard pointing devices. A prospective observational study was performed on academic personnel, with a comparative follow-up study utilizing OB/GYN residents. Two tasks were designed, a maze task measuring the accuracy of pointing and a point task measuring the speed. A laparoscopic video monitor 12 was placed at a height of 145 cm and distance of 120 cm. Participants stood 15° offset from perpendicular to the monitor while using the devices. Testing was performed in a prototyping lab and a simulated operating room for academic personnel and residents, respectively. Twenty-three academic personnel volunteered for the initial study. The follow-up comparative study utilized ten resident volunteers. Individuals took 15 minutes to complete the tasks. Follow-up surveys were conducted.

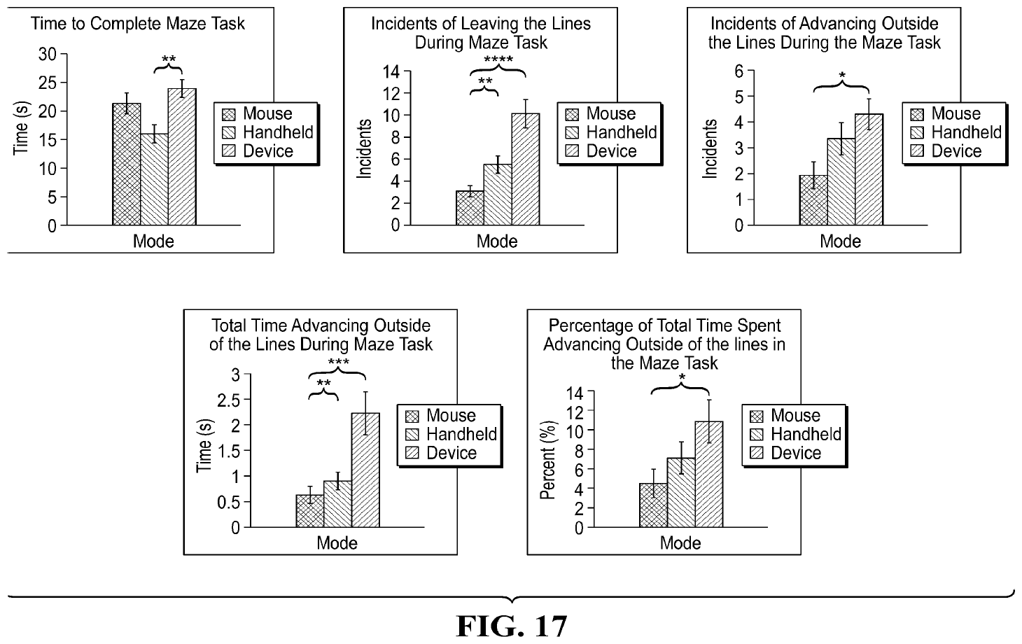

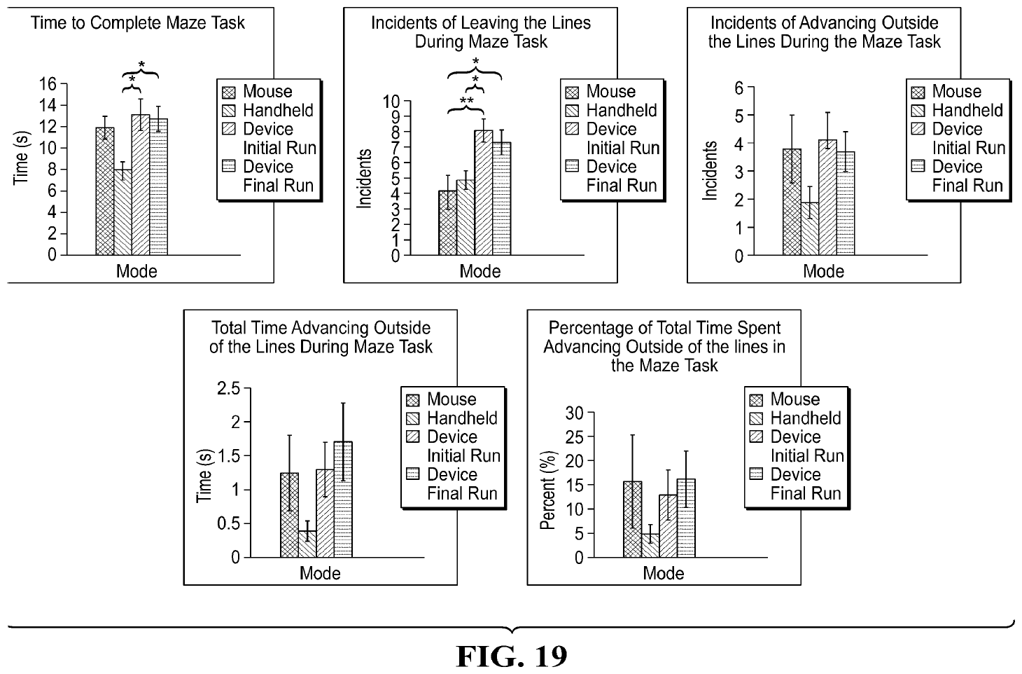

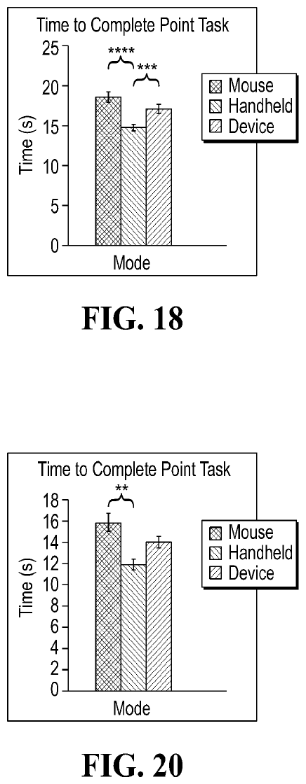

Participants were taken through a series of non-inferiority tests using the apparatus 10 disclosed herein, a handheld laser pointer, and a mouse. The maze task (FIGS. 17 and 19) involved using these different devices to complete a simple maze, and the point task (FIGS. 18 and 20) involved pointing at dots as they appeared randomly on the video monitor 12. For the maze task, participants were timed and their progress was recorded to track errors. Analysis demonstrated no significant difference in errors by the residents between the disclosed apparatus 10 and the laser pointer (p=0.05). For the point task, subjects were timed. No significant difference was found in time by the residents between the disclosed apparatus 10 and a handheld laser pointer or mouse. Overall, the residents performed the tasks faster than academic personnel. Survey results indicated no significant difference between the devices in comfort and perceived performance for both groups.

The apparatus 10 disclosed herein allows laparoscopic surgeons (“teachers”) to communicate with trainees (“learners”) more clearly without the use of occupied hands. Data from the studies described above indicates that the disclosed apparatus 10 can perform comparably to handheld teaching devices.

While the disclosed apparatus 10 may be designed for a laparoscopic teaching setting, it may have general application to any situation where the surgery or medical procedure involves more than one pair of hands and where communication between a physician and an assistant is needed. Accordingly, while described herein with respect to laparoscopic surgery, the apparatus 10 may also be suitable for use in other surgical settings and during other medical procedures which involve coordination between a physician and an assistant. In one example, the apparatus 10 could be used during gynecology procedures, such as hysterectomy, ovarian cystectomy, and excision of endometriosis. In another example, the apparatus 10 could be used during general surgery, such as during appendectomy, cholecystectomy, bariatric surgery, and colorectal surgery. Other surgical and medical procedures in which the disclosed apparatus 10 may be utilized are also fully contemplated.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. An apparatus for providing training for a learner by a teacher during laparoscopic surgery, the apparatus comprising:
a housing including a first glasses arm, the housing configured to be mounted to a pair of glasses by the first glasses arm;
a light source mounted on the housing and configured to generate a light beam; and
a control module disposed within the housing in electrical communication with the light source, the control module including a power source for providing power to the light source;
wherein when the pair of glasses is worn by the teacher, a direction of the light beam generated by the light source corresponds to a head position of the teacher to indicate a location for the learner.

2. The apparatus of claim 1, wherein the light source includes a laser pointer.

3. The apparatus of claim 1, further comprising a foot pedal in wireless communication with the control module for hands-free operation of the light source.

4. The apparatus of claim 1, further comprising a switch disposed within the housing in electrical communication with the light source.

5. The apparatus of claim 1, wherein the first glasses arm includes a front mounting portion including mounting pins extending therefrom for removably and pivotally mounting the housing to the pair of glasses.

6. The apparatus of claim 1, wherein the first glasses arm includes an upper mounting portion including at least one tab having a channel configured to at least partially receive the light source.

7. The apparatus of claim 1, wherein the housing includes a first cavity for mounting the control module, and the apparatus further includes a cover removably connectable to the housing for enclosing the first cavity.

8. An apparatus for providing training for a learner by a teacher during laparoscopy surgery, the apparatus comprising:
a pair of glasses including a lens portion and a frame portion surrounding the lens portion;
a housing including a first glasses arm, the housing configured to be mounted to the frame portion by the first glasses arm;
a laser pointer mounted on the housing and configured to generate a light beam; and
a control module disposed in a first cavity within the housing in electrical communication with the laser pointer, the control module including a power source for providing power to the laser pointer;
wherein the pair of glasses is arranged to be worn by the teacher such that a direction of the light beam generated by the laser pointer corresponds to a head position of the teacher to indicate a location for the learner.

9. The apparatus of claim 8, further comprising a foot pedal in wireless communication with the control module for hands-free operation of the laser pointer.

10. The apparatus of claim 8, wherein the first glasses arm includes a front mounting portion including mounting pins extending therefrom for removably and pivotally mounting the housing to the frame portion.

11. The apparatus of claim 8, wherein the first glasses arm includes an upper mounting portion including at least one tab having a channel configured to at least partially receive the laser pointer.

12. The apparatus of claim 11, wherein the housing includes a hollow projection member adjacent the upper mounting portion and including a conduit connected to the first cavity for routing wiring from the laser pointer to the first cavity.

13. The apparatus of claim 8, further comprising a switch disposed within the housing in electrical communication with the laser pointer.

14. The apparatus of claim 8, further comprising a cover removably connectable to the housing for enclosing the first cavity.

15. An apparatus for providing training for a learner by a teacher during laparoscopic surgery, the apparatus comprising:
a pair of glasses including a lens portion and a frame portion surrounding the lens portion;
a housing including a first glasses arm having a front mounting portion with mounting pins extending therefrom for removably and pivotally mounting the housing to the frame portion;
a laser pointer mounted on the first glasses arm and configured to generate a light beam;
a control module disposed within a first cavity of the housing in electrical communication with the laser pointer, the control module including a power source for providing power to the laser pointer; and
a foot pedal in wireless communication with the control module for hands-free control of the laser pointer;
wherein the pair of glasses is arranged to be worn by the teacher such that a direction of the light beam generated by the laser pointer corresponds to a head position of the teacher to indicate a location for the learner.

16. The apparatus of claim 15, wherein the first glasses arm includes an upper mounting portion including at least one tab having a channel configured to at least partially receive the laser pointer.

17. The apparatus of claim 16, wherein the housing includes a hollow projection member adjacent the upper mounting portion and including a conduit connected to the first cavity for routing wiring from the laser pointer to the first cavity.

18. The apparatus of claim 15, further comprising a switch disposed within the housing in electrical communication with the laser pointer.

19. The apparatus of claim 15, further comprising a cover removably connectable to the housing for enclosing the first cavity.

20. The apparatus of claim 15, further comprising a head strap arranged to be connected to the pair of glasses.
